(19) United States
(12) Patent Application Publication
(10) Pub. No.: US 2006/0294610 A1
(43) Pub. Date: Dec. 28, 2006

(54) Alpha1-3 galactosyltransferase gene and promoter

(75) Inventor: Chihiro Koike, Pittsburgh, PA (US)

Correspondence Address: KING & SPALDING LLP, 1180 PEACHTREE STREET, ATLANTA, GA, 30309, US

(73) Assignee: University of Pittsburgh of the Commonwealth System of Higher Education, Pittsburgh, PA (US)

(21) Appl. No.: 11/434,628

(22) Filed: May 16, 2006

Related U.S. Application Data
(63) Continuation of application No. 10/125,994, filed on Apr. 19, 2002, now abandoned.
Application No. 10/125,994 is a continuation of application No. PCT/US00/29139, filed on Oct. 20, 2000.
(60) Provisional application No. 60/161,092, filed on Oct. 22, 1999.
(60) Provisional application No. 60/227,951, filed on Aug. 25, 2000.

(51) Int. Cl. A01K 67/027

(52) U.S. Cl. 800/17; 800/14; 800/15; 800/18; 435/455; 435/325

(57) ABSTRACT

The present invention provides a recombinant expression cassette comprising an α1-3 galactosyltransferase promoter operably linked to a polynucleotide for expression. The invention also provides a recombinant mutating cassette comprising a region of homology to an α1-3 galactosyltransferase genomic sequence. The cassettes can be employed to express foreign genes or to disrupt the native α1-3 galactosyltransferase genomic sequence, particularly within an animal. Thus, the invention also provides transgenic animals and methods for their production and use.

### CROSS-REFERENCE TO RELATED APPLICATIONS

This is a continuation of co-pending international patent application PCT/US00/29139, which designates the United States and which was filed on Oct. 20, 2000 claiming priority to U.S. Provisional Application for Patent 60/161,092, which was filed Oct. 22, 1999, and also to U.S. Provisional Application for Patent 60/227,951, which was filed Aug. 25, 2000.

### TECHNICAL FIELD OF THE INVENTION

This invention relates to the α1-3 galactosyltransferase gene, promoters therefor, and the use thereof to create transgenic animals.

### BACKGROUND OF THE INVENTION

The current shortage of acceptable organs for transplantation is a major health concern. Because the demand for acceptable organs exceeds the supply, many people die each year while waiting for organs to become available. To help meet this demand, research has been focused on developing alternatives to allogenic transplantation. Thus, for example, dialysis has been available to patients suffering from kidney failure, artificial heart models have been tested, and other mechanical systems have been developed to assist or replace failing organs. Such approaches, however, are quite expensive, and the need for frequent and periodic access to such machines greatly limits the freedom and quality of life of patients undergoing such therapy.

Xenograft transplantation represents a potentially attractive alternative to artificial organs for human transplantation. The potential pool of nonhuman organs is virtually limitless, and a successful xenograft transplantation would not render the patient virtually tethered to machines as is the case with artificial organ technology. Host rejection of such cross-species tissue, however, remains a major concern in this area. Some noted xenotransplants of organs from apes or old-world monkeys (e.g., baboons) into humans have been tolerated for months without rejection. However, such attempts have ultimately failed due to a number of immunological factors. Even with heavy immunosupression to suppress hyperaccute rejection, a low-grade innate immune response, attributable in part to failure of complement regulatory proteins (CRPs) within the graft tissue to control activation of heterologous complement on graft endothelium, ultimately leads to destruction of the transplanted organs (see e.g., Starzl, Immunol. Rev., 141, 213-44 (1994)). In an effort to develop a pool of acceptable organs for xenotransplantation into humans, researchers have engineered animals producing human CRPs, an approach which has been demonstrated to delay, but not eliminate, xenograft destruction in primates (McCurry et al., Nat. Med., 1, 423-27 (1995); Bach et al., Immunol Today, 17, 379-84 (1996)).

In addition to complement-mediated attack, human rejection of discordant xenografts appears to be mediated by a common antigen: the galactose-α(1,3)-galactose (gal-α-gal) terminal residue of many glycoproteins and glycolipids (Galili et al., Proc. Nat. Acad. Sci. (USA), 84, 1369-73 (1987); Cooper et al., Immunol Rev., 141, 31-58 (1994); Galili et al., Springer Sem. Immunopathol, 15, 155-171 (1993); Sandrin et al., Transplant Rev., 8, 134 (1994)). This antigen is chemically related to the human A, B, and O blood antigens, and it is present on many parasites and infectious agents, such as bacteria and viruses. Most mammalian tissue also contains this antigen, with the notable exception of old world monkeys and apes (including humans) (see Joziasse et al., J. Biol. Chem., 264, 14290-97 (1989) and references cited therein)). The antigen is highly immunogenic in humans, and many individuals show significant levels of circulating IgG with specificity for gal-α-gal carbohydrate determinants (see, e.g., Galili et al., J. Exp. Med., 162, 573-82 (1985), Galili et al., Proc. Nat. Acad. Sci. (USA), 84, 1369-73 (1987)). Thus, in hopes of better understanding barriers to xenotransplantation, recent attention has turned to the enzyme mediating the formation of gal-α-gal moieties: α1-3 galactosyltransferase.

The expression of α1-3 galactosyltransferase is regulated both developmentally and in a tissue-specific manner. The cDNA for this enzyme has been isolated from many species, including pigs (Hoopes et al., poster presentation at the 1997 Xenotransplantation Conference, Nantes France; Katayama et al., J. Glycoconj., 15(6), 583-99 (1998); Sandrin et al., Xenotransplantation, 1, 81-88 (1994), Strahan et al., Immunogenics, 41, 101-05 (1995)), mice (Joziasse et al., J. Biol. Chem., 267, 5534-41 (1992)), and cows (Joziasse et al., J. Biol. Chem., 264, 14290-97 (1989). While authors have proposed to eliminate the gene from xenograft donor animals (Sandrin et al. (1994), supra; U.S. Pat. No. 5,821,117 (Sandrin et al.)), gene knock-out procedures generally require knowledge of the genomic structure and sequence beyond the cDNA of a given gene. The genomic organization of the mouse α1-3 galactosyltransferase homologue has been deduced (Joziasse et al., J. Biol. Chem., 267, 5534-41 (1992)), and human homologues are known to be inactive pseudogenes (see Joziasse et al., J. Biol. Chem., 266, 6991-98 (1991); Larsen et al., J. Biol. Chem., 265, 7055-61 (1990)). However, the genomic organization of an α1-3 galactosyltransferase homologue from a species that could serve as a xenograft donor for human recipients has yet to be deduced, and no promoter for any α1-3 galactosyltransferase homologue gene is known. As such, there exists a need for methods and reagents for facilitating xenotransplantation between species, particularly between species exhibiting differential expression of the gal-α-gal epitope.

### BRIEF SUMMARY OF THE INVENTION

The present invention provides a recombinant expression cassette comprising an α1-3 galactosyltransferase promoter operably linked to a polynucleotide for expression. The invention also provides a recombinant mutating cassette comprising a region of homology to an α1-3 galactosyltransferase genomic sequence. The cassettes can be employed to express foreign genes or to disrupt the native α1-3 galactosyltransferase genomic sequence, particularly within an animal. Thus, the invention also provides transgenic animals and methods for their production and use. These aspects of the invention, as well as additional inventive features, will be apparent from the accompanying drawing, sequence listing, and the following detailed description.

### BRIEF DESCRIPTION OF THE DRAWINGS

FIGS. 1A through 1I depict the genomic organization porcine α1-3 galactosyltransferase gene. FIG. 1A depicts all introns and exons of the gene, indicating the size of the respective elements. FIGS. 1B through 1I depict alternatively spiced variants isolated from pig aortic endothelial cells.

FIG. 2 depicts the organization of a portion of the porcine α1-3 galactosyltransferase promoter.

FIG. 3 depicts the organization of the alternate splicing patterns observed in the expression of the human untranslated α1,3 galactosyltransferase pseudogene.

### DETAILED DESCRIPTION OF THE INVENTION

In a first aspect, the present invention provides a recombinant expression cassette in which an α1-3 galactosyltransferase promoter is operably linked to a polynucleotide for expression. The expression cassette is “recombinant” in that within the inventive cassette, the polynucleotide for expression is other than one encoding α1-3 galactosyltransferase. The promoter and the polynucleotide are “operably linked” in that an event at the promoter (e.g., binding of cellular transcription factors and other DNA binding proteins) precipitates expression (i.e., transcription) of the polynucleotide. So long as this operable linkage is maintained, the cassette can include elements other than the α1-3 galactosyltransferase promoter and the polynucleotide for expression. For example, the cassette can contain polyadenylation sequences, repressors, enhancers, splice signals, signals for secretion (see, e.g., U.S. Pat. No. 4,845,046 and European Patent EP-B-319,641), etc. Moreover, the expression cassette can include more than one polynucleotide operably linked to the α1-3 galactosyltransferase promoter, (e.g., multiple coding sequences separated by internal ribosome entry sites).

The α1-3 galactosyltransferase promoter can be derived from any species normally expressing the gene. Thus, for example, the promoter can be derived from the bovine, porcine, or murine α1-3 galactosyltransferase genes. Examples of such promoters are set forth at SEQ ID Nos:1-6. However, the α1-3 galactosyltransferase promoter is not limited to one of these sequences, as it can be an active fragment of one of these sequences or a derivative of one of these sequences having one or more mutations (e.g., point mutations, substitutions, insertions, deletions, etc.). Furthermore, given the instant disclosure, it is within the ordinary skill of the art to assay regions of the α1-3 galactosyltransferase gene unrelated to SEQ ID NOs:1-6 for promoter activity, and the inventive expression cassette can include any α1-3 galactosyltransferase promoters so identified. Suitable promoters can be readily identified by construction an expression cassette in which the derivative sequence is operably linked to a desired reporter gene (e.g., RNA for detection by Northern hybridization, or DNA encoding CAT, luciferase, green-fluorescent peptide, β-galactosidase, etc.) and introducing the cassette into a suitable environment for transcription and (where appropriate) translation. Subsequently, promoter activity is detected by assaying for the presence of the reporter by standards methods (e.g., Northern hybridization, Southern hybridization, enzymatic detection, immunohistochemistry, etc.).

Within the expression cassette, the α1-3 galactosyltransferase promoter can be operably linked to any desired coding polynucleotide. Generally, where expression of a given gene or factor is desired, the skilled artisan will be in possession of the sequence of the coding polynucleotide. Thus, the polynucleotide can be expressed as a bioactive RNA molecule (e.g., an antisense RNA or a ribozyme). Alternatively, the polynucleotide can encode a protein of interest, and in this embodiment, the polynucleotide can be or comprise cDNA or genomic DNA.

Where the polynucleotide encodes a protein, any desired protein can be so encoded, and it need not be syngenic to the species from which the promoter is derived. Thus, for example, the cassette can be employed in animals to produce proteins facilitating growth or bulking of the animal (e.g., bovine or human growth factor) for conferring resistance to disease or parasites. Other encoded proteins can be enzymes such as sulfo- or glycosyltransferases, (e.g., a fucosyltransferase, a galactosidase, a galactosyltransferase, a, a β-acetylgalactosaminyltransferase, an N-acetylglycosaminyltransferase, an N-acetylglucosaminyltransferase, a sialyltransferase, etc.). Where the expression cassette is employed to generate tissue or organs for xenotransplantation into an organism lacking gal-α-gal antigens (as described below), preferably the polynucleotide encodes a Type I fucosyltransferase, a Type II fucosyltransferase, an α2-3 sialyltransferase, or an α2-6 sialyltransferase from any species, the coding sequences of which are known (see, e.g., Larsen et al., Proc. Nat. Acad. Sci. (USA), 87, 6674-78 (1990); Kelly et al., J. Biol. Chem., 270(9), 4640-49 (1995), J. Biol. Chem., 268(30), 22782-87 (1993), Weinstein et al., J. Biol. Chem., 262(36), 17735-43 (1987)).

The expression cassette can be constructed by conventional methods of molecular biology (e.g., direct cloning by ligation, site specific recombination using recombinases, such as the flp recombinase or the cre-lox recombinase system (reviewed in Kilby et al. Trends Genet., 9, 413-21 (1993)), homologous recombination, and other suitable methods). Typically, the promoter sequence is introduced into a vector 5′ (i.e., “upstream”) of the coding polynucleotide and any other elements (e.g., ribosome entry sites, polyadenylation sequences, etc.), after which the construct is subcloned and grown in a suitable host organism (e.g., yeast, bacteria, etc.) from which it can be isolated or substantially (and typically completely) purified by standard methods. Thus, the invention provides a vector (preferably an isolated or substantially purified vector) including a recombinant expression cassette as set forth above. Such a vector can be any desired type of vector, such as naked DNA vectors (e.g., oligonucleotides or plasmids); viral vectors (e.g., adeno-associated viral vectors (Berns et al., Ann. N.Y. Acad. Sci., 772, 95-104 (1995)), adenoviral vectors (Bain et al., Gene Therapy, 1, S68 (1994)), bacteriaphages, baculovirus vectors (see, e.g., Luckow et al., Bio/Technology, 6, 47 (1988)), herpesvirus vectors (Fink et al., Ann. Rev. Neurosci., 19, 265-87 (1996)), packaged amplicons (Federoffet al., Proc. Nat. Acad Sci. USA, 89, 1636-40 (1992)), papilloma virus vectors, picomavirus vectors, polyoma virus vectors, retroviral vectors, SV40 viral vectors, vaccinia virus vectors) or other vectors (e.g., a cosmid, a yeast artificial chromosome (YAC), etc.). Of course, the vector can (and typically does) contain elements in addition to the expression cassette that are appropriate to the type of vector (e.g., origins of replication, marker genes, genes conferring resistance to antibiotics, etc.). The insertion of the expression cassette can disrupt one or more of these elements, if desired, or the cassette can be inserted between genetic elements to minimize perturbation of the backbone vector.

Where the vector is a viral vector, preferably it is replication incompetent. Thus, for example, an adenoviral vector preferably has an inactivating mutation in at least the E1A region, and more preferably in region E1 (i.e., E1A and/or E1B) in combination with inactivating mutations in region E2 (i.e., E2A, E2B, or both E2A and E2B), and/or E4 (see, e.g., International Patent Application WO 95/34671). An AAV vector can be deficient in AAV genes encoding proteins associated with DNA or RNA synthesis or processing or steps of viral replication (e.g., capsid formation) (see U.S. Pat. Nos. 4,797,368, 5,354,768, 5,474,935, 5,436,146, and 5,681,731). Where the vector is a retroviral vector, the cis-acting encapsidation sequence (E) essential for virus production in helper cells can be deleted upon reverse transcription in the host cell to prevent subsequent spread of the virus (see, e.g., U.S. Pat. No. 5,714,353). Where the vector is a herpesvirus, inactivation of the ICP4 locus and/or the ICP27 cassette renders the virus replication incompetent in any cell not complementing the proteins (see, e.g., U.S. Pat. No. 5,658,724, see also DeLuca et al., J. Virol., 56, 558-70 (1985); Sarnaniego et al., J. Virol., 69(9), 5705-15 (1996)).

To use the inventive recombinant expression cassette, it is introduced into a eukaryotic cell in a manner suitable for the cell to express the coding polynucleotide. A vector harboring the recombinant expression cassette is introduced into a eukaryotic cell by any method appropriate for the vector employed, which generally are well-known in the art. Thus, plasmids are transferred by methods such as calcium phosphate precipitation, electroporation, liposome-mediated transfection, microinjection, viral capsid-mediated transfer, polybrene-mediated transfer, protoplast fusion, etc. Viral vectors are best transferred into the cells by infecting them.

Depending on the type of vector, it can exist within the cell as a stable extrachromosomal element (which can even be heritable, see e.g., Gassmann, M. et al., Proc. Natl. Acad. Sci. (USA), 92, 1292 (1995)) or it can integrate into the host cell's chromosomes. Thus, the invention provides a chromosome including a recombinant expression cassette such as described above, as well as a cell including such a cassette (and such a chromosome). The α1-3 galactosyltransferase promoter of the expression cassette can be native to such a cell or chromosome, or it can be exogenous to the cell or chromosome. Where the promoter is native to the cell or chromosome, preferably the polynucleotide for expression within the cassette (the non-native polynucleotide) displaces the operable linkage between the native polynucleotide encoding α1-3 galactosyltransferase such that it is no longer operably linked to the native α1-3 galactosyltransferase promoter. Such displacement can be accomplished where the non-native polynucleotide is cloned between the promoter and the native polynucleotide (i.e., upstream of the native polynucleotide), especially where the non-native polynucleotide contains one or more transcriptional termination signals (preferably in all three putative reading frames). Of course, the non-native polynucleotide also can be introduced into the locus such that it destroys the native exon/intron boundaries and/or introduces inactivating mutations (e.g., deletions, insertions, frame-shifts, etc.) into the native coding sequence.

Preferably, the transgenic cell presents a suitable microenvironment for the coding polynucleotide within the expression cassette to be expressed. In many instances, the transgenic cells can be used to study the tissue specificity, dynamics, and kinetics of the promoter, for example by assaying for the expression of the polynucleotide within the cells. However, as the absence of activity is as useful as the presence of promoter activity in these contexts, any cell can be employed for such purposes; such a cell can be in vivo or in vitro. Preferably, the cell is derived from a species syngenic to the source of the promoter so that, by virtue of the properties of the α1-3 galactosyltransferase promoter present within the expression cassette, the polynucleotide within the cassette is expressed within such transgenic tissues, organs, or animals with the same kinetics and tissue specificity as the native α1-3 galactosyltransferase gene in wild-type animals. Where the cells are in vivo, they are typically cells of a mammal (e.g., human cells), and can be any type of cells. Suitable cells for use in vitro include yeast, protozoa (e.g., T. cruzi epimastigotes), cells derived from any mammalian species (e.g., VERO, CV-1, COS-1, COS-7, CHO-K1, 3T3, NIH/3T3, HeLa, C1271, BS-C-1 MRC-5, etc.), insect cells (e.g., Drosophila Snyder cells), or other such cells. In other applications, the cell can be employed to construct transgenic tissues, organs, or animals, as described below, in which case the cell typically is a spermatozoon, ovum, zygote, primordial germ cells, or embryonic stem cell.

In another embodiment, the invention provides a method of mutating a region of a chromosome comprising an α1-3 galactosyltransferase gene. In accordance with the inventive method, a recombinant mutating cassette comprising a region of homology to the α1-3 galactosyltransferase gene is recombined with a chromosome which has an α1-3 galactosyltransferase gene such that homologous recombination occurs between the cassette and the chromosome. As a result of the homologous recombination, a mutation is introduced into the native α1-3 galactosyltransferase chromosomal gene sequence. Thus, the final step of the method involves screening for successful recombination.

The inventive method employs a recombinant mutating cassette including at least a first region of homology to an α1-3 galactosyltransferase genomic sequence, and the invention provides such a cassette. Within such a cassette, this first region of homology is adjacent to either to at least one polynucleotide for insertion or to a second region of homology. The mutating cassette is “recombinant” in that neither the second region of homology nor the polynucleotide for insertion is adjacent to the first α1-3 galactosyltransferase genomic sequence in its native state (i.e., within a chromosome).

The insertion cassette can include more than one polynucleotide for insertion and/or more than one region of homology to all or a portion of the α1-3 galactosyltransferase genomic sequence. Indeed, where the cassette includes a region for insertion, preferably it has at least two regions of homology flanking the region for insertion. Where more than one region of homology is present, whether adjacent to each other or flanking a region for insertion, the cassette can be used to replace any span of the target chromosomal genomic sequence that lies between the two homologous chromosomal regions. Where multiple regions of homology are present, they should generally be arrayed in the same 5′ to 3′ orientation relative to one another.

A region of homology can be homologous to any portion of the genomic sequence of an α1-3 galactosyltransferase gene or the antisense strand thereof. The region can be homologous to the gene of any desired species, such as those discussed above, and it can be homologous to an intron, an exon, a promoter sequence, or any other desired sequence from the genomic DNA. To this end, regions of homology can be selected from the promoter sequences disclosed in SEQ ID NOs:1-6. Alternatively (or additionally) a region of homology can be selected from a portion of the genomic sequence from an α1-3 galactosyltransferase homologue. In this light, some of the murine sequences have been published (see, e.g., Joziasse et al., J. Biol. Chem., 267, 5534-41 (1992)), and additional portions are set forth as SEQ ID NOs: 17-25. Portions of the porcine genomic sequence are disclosed herein as SEQ ID NOs: 7-16. Portions of the human α1,3 galactosyltransferase pseudogene genomic sequences are set forth at SEQ ID NOs: 35-42, and various (untranslated) human cDNA transcripts are set forth as SEQ ID NOs: 27-34, and those from Rhesus monkeys are set forth at SEQ ID NOs: 43-44. These sequences disclosed herein, as well as the published murine sequences, include the intron/exon boundaries from which one of skill in the art can isolate additional intronic genomic sequences by techniques such as genome walking, 5′ RACE, 3′ RACE, etc.

A region of homology to the genomic sequence of an α1-3 galactosyltransferase gene need not be an exact complement to the genomic sequence; however, the region must be sufficiently homologous to the α1-3 galactosyltransferase gene to permit homologous recombination between the cassette and the genomic DNA in vivo. Indeed, in some embodiments (e.g., for introducing point mutations into the genomic sequence), a region of homology preferably contains some mismatched bases. Thus, typically, the region of homology will bear at least about 75% homology to a portion of the α1-3 galactosyltransferase gene or its antisense strand (such as at least about 85% homology to a portion of the α1-3 galactosyltransferase gene or its antisense strand), and more typically the region of homology will bear at least about 90% homology to a portion of the α1-3 galactosyltransferase gene or its antisense strand (such as at least about 95% or even at least about 97% homology to a portion of the α1-3 galactosyltransferase gene or its antisense strand). Any commonly employed method (e.g., BLAST database searching) for calculating percent homology can be used to select a suitable region of homology. Similarly, while the length of the region of homology is not critical, it should be sufficiently long to facilitate homologous recombination between the cassette and the genomic DNA in vivo. Thus, typically the region of homology will be at least about 50 nucleotides long (such as at least about 75 or 100 bases long), and more typically it will be at least several hundred bases long (such as at least about 250, 500, or even 750 bases long). Indeed, in many applications, the region of homology preferably is several thousand bases long to maximize the likelihood of homologous recombination in vivo. The ideal length of a region of homology depends in part on the number of such regions within the cassette—where one or few regions of homology are present, they should be longer to facilitate recombination between the cassette and the genomic DNA; conversely, where the cassette contains several regions of homology, they can be shorter without reducing the likelihood of recombination events.

Where present within the cassette, a region for insertion can be or comprise any DNA which is desired to be introduced into the genomic sequence of an α1-3 galactosyltransferase gene. Thus, the region can comprise genetic regulatory elements (e.g., enhancers, promoters, repressors, etc., the sequences of which are known) or consensus binding sites for DNA-binding proteins (e.g., restriction endonucleases, transcription factors, etc.). In many applications, a region for insertion can comprise a polynucleotide for expression, such as those set forth above, or even expression cassettes. A preferred polynucleotide for insertion is an expression cassette for expressing a positive marker flanked by FRT sites, thus facilitating the identification of chromosomes into which the polynucleotide for insertion has integrated as well as excision of the cassette.

The mutating cassette can be constructed by any desirable molecular techniques, and typically, the mutating cassette will be engineered within a vector, such as those set forth above. Typically, the vector is a gene transfer vector suitable for introducing the cassette into a host cell. In addition to the region(s) of homology and the polynucleotide for insertion elements, the mutating cassette can have other components, such as, for example, an expression cassette, a region of homology to other genes or chromosomal regions, a polyadenylation sequence, etc., and it is preferred that the insertion cassette comprises a cassette for expressing at least one marker gene (which may be or comprise the polynucleotide for insertion). Such a marker can be either positive (conferring a visible phenotype to the cells) or negative (killing cells or rendering non-recombinant cells growth-impaired), and both can be used in conjunction. Examples of such positive and negative selection markers are the neosporin resistance (neo^{R}) gene, the hydromycin resistance (hyg^{R}) gene, and a thymidine kinase gene (e.g., HSV tk); other suitable markers are known in the art (see, e.g., Mansour et al., Nature, 336, 348-52 (1988); McCarrick et al., Transgen. Res., 2, 183-90 (1993)). A marker gene sequence can be bordered at both ends by FRT DNA elements, and/or with stop codons for each of the three putative reading frames being inserted 3′ to the desired DNA sequence. Presence of the FRT elements permits the marker to be deleted from the targeted chromosome, and the stop codons ensure that the α1,3 galactosyltransferase gene remains inactivated following deletion of the selectable marker, if inactivation is the desired result of the use of the mutating cassette. The relative orientations of the positive and negative selectable markers are not critical. However, where a positive marker is employed, it should be located between regions of homology, while any negative marker should be outside the regions of homology, either 5′ or 3′ to those regions.

In accordance with the inventive method, homologous recombination occurs between the α1-3 galactosyltransferase genomic chromosomal DNA and the region (or regions) of homology in the mutating cassette. Where more than one region of homology is present in the cassette, any portion of the genome lying between the homologous target sequences is replaced by whatever sequence lies between the regions of homology in the cassette. Thus, where the mutating cassette contains a region for insertion flanked by two regions of homology, it will be introduced into the genomic sequence adjacent to the sites of homology, replacing that portion of the genomic sequence. Of course, where the two flanking regions of homology are normally adjacent to each other in the chromosomal sequence, the region for insertion is introduced into the chromosome without replacing any native sequence. Similarly, where no region for insertion is present within the cassette, that portion of the chromosome lying between the two regions of homology in the cassette is deleted as a result of the recombination events. Where the cassette contains a region of homology that differs slightly from the homologous sequence within the genome, it can be employed to introduce point mutations into the genomic sequence.

While the recombination event can occur in vitro, typically such homologous recombination occurs within a host cell between an exogenous vector containing the cassette and a chromosome within the host cell containing an α1-3 galactosyltransferase genomic sequence. Thus, the present invention provides a cell harboring a mutating cassette, as described above. The vector can be introduced into the host cell by any appropriate method, such as set forth above. Commonly, however, the vector is introduced into small cells (e.g., embryonic stem cells) by electroportation and into large cells (e.g., ova or zygotes) by microinjection. Where microinjection is employed, the vector preferably is injected directly into a nucleus or pronucleus of the cell.

The last step in the method is to screen for successful recombination events. Any assay to detect such events can be employed in the context of the inventive method. In accordance with one such assay, chromosomal DNA is screened by PCR or Southern hybridization. For example, where the mutating cassette is designed to delete a portion of the α1-3 galactosyltransferase genomic sequence, the absence of signal using a probe or primer directed against the region to be deleted indicates a positive recombination event. Conversely, where the cassette includes a region for insertion, a positive result using a probe or primer directed against the region for insertion is indicative of a positive recombination event. Of course, the chromosomal DNA can be sequenced to confirm the correct insertion/deletion/replacement. Where recombination is directed within cells, the events can be screened by assaying for any markers present in the mutating cassette.

By employing the inventive method, one of skill in the art can use the inventive mutating cassette to introduce targeted deletions, insertions, or replacement mutations into any predefined site within the α1-3 galactosyltransferase genomic sequence. Any desired amount or portion of the gene can be thus deleted, which can lead to complete inactivation of the gene. For introducing inactivating mutations into the gene, preferably at least one region of homology is selected to recombine with the promoter (to inactivate it) or exons 4-9, which contain the coding sequences. Similarly, the inventive method can introduce functional expression cassettes in place of the α1-3 galactosyltransferase gene, which can be under the control of the native α1-3 galactosyltransferase promoter or an exogenous promoter within the cassette (especially where the native α1-3 galactosyltransferase promoter is destroyed). Thus, the present invention provides a recombinant chromosome containing such a mutation, and a recombinant cell comprising such a chromosome.

As mentioned above, the invention provides recombinant cells and chromosomes comprising a recombinant expression cassette comprising an α1-3 galactosyltransferase promoter or a mutating cassette, as described above. Indeed, as a result of using these reagents and methods, the invention also provides a cell having a mutant α1-3 galactosyltransferase genomic sequence, as described above. While any cell having such exogenous genetic sequences is within the scope of the invention, preferably the cells are suitable for constructing a recombinant animal, and are most preferably totipotent cells. Thus, preferred cells are embryonic stem (ES) cells, ova, primordial germ cells (PGCs), and zygotes. ES cells and PGCs are especially preferred because such cells can be obtained and cultured in relatively large numbers relative to ova and zygotes. Using such cells, a transgenic animal having an expression cassette comprising an α1-3 galactosyltransferase promoter or a disruption in this gene can be constructed by methods known in the art (see e.g., U.S. Pat. No. 5,850,004 (MacMicking et al.), U.S. Pat. No. 5,942,435 (Wheeler), U.S. Pat. No. 5,523,226 (Wheeler), and U.S. Pat. No. 5,175,383; White et al., Transplant. Int., 5, 648-50 (1992); McCurry et al., Nat. Med., 1, 423-427 (1995); Hoganet al., Manipulating the Mouse Embryo, Cold Spring Harbor Laboratory, Cold Spring Harbor, N.Y. (1986); Hammeret al., Nature, 315, 680 (1985); Murrayet al., Reprod. Fert. Devl., 1, 147, (1989); Purselet al., Vet. Immunol. Histopath., 17, 303 (1987); Rexroadet al., J. Reprod. Fert., 41, (suppl.), 119 (1990); Rexroadet al., Molec. Reprod. Devl., 1, 164 (1989); Simonset al., BioTechnology, 6, 179 (1988); Vizeet al., J. Cell. Sci., 90, 295 (1988); Wagner, J. Cell. Biochem., 13B (suppl.), 164 (1989); Thomas et al., Cell, 51, 503 (1987); Capecchi, Science, 244, 1288 (1989); Joyner et al., Nature, 338, 153 (1989); Ausubelet al., Cur. Prot. Mol. Biol., John Wiley & Sons (1987)).

Where ova and zygotes are employed, after the introduction of the cassette, they can be implanted into surrogate mothers to develop into adult animals. Where ES cells or PGCs are employed, after the introduction of the cassette, they typically are further manipulated (e.g., by injection into a blastocyst or morula, co-culture with a zona pellucida-disrupted morula, fusion with an enucleated zygote, etc.) such that their mitotic descendants are found in a developing embryo. Such an embryo typically is a chimera composed of normal embryonic cells as well as mitotic descendants of the introduced ES cells or PGCs. Alternatively, the genome of an ES cell or PGC can be incorporated into an embryo by fusing the ES cell/PGC with an enucleated zygote to create a non-chimeric embryo in which all nuclei are mitotic descendants of the fused ES cell/PGC nucleus. In any event, to produce a transgenic animal, the embryo or zygote is implanted into a pseudopregnant animal, which, after suitable gestation, gives birth to an animal containing the mutant chromosome containing the cassette in its germ line (if a chimera) or possibly all of its cells. Of course, as mentioned above, where the animal is engineered to include a non-mutating expression cassette, it can be inherited as an extrachromosomal plasmid (Gassmann, M. et al., supra)). However constructed, the presence of the recombinant allele can be confirmed by performing Northern hybridization or rt-PCR on RNA isolated from the animal in question.

After birth and sexual maturation, a chimeric animal can be mated to generate a heterozygous animal comprising a disrupted α1-3 galactosyltransferase gene or recombinant expression cassette (integrated or extrachromosomal) including a α1-3 galactosyltransferase promoter. Heterozygotes can be crossed to produced a homozygous strain. Such animals having a recombinant expression cassette including an α1-3 galactosyltransferase promoter, as discussed above, will express the polynucleotide for expression of such cassette within the same tissue types and with the same kinetics as a wild-type animal of the same species and strain expresses the α1-3 galactosyltransferase gene. Of course, homozygous transgenic animals of the present invention having a disruption in the α1-3 galactosyltransferase gene will produce altered forms of the protein or no functional protein at all. Desirably, the phenotype of such “knock out” animals relative to an animal having a wild type α1-3 galactosyltransferase gene is a markedly increased time of survival of cells isolated or derived from the transgenic animal in the presence of human serum, which can be assessed by any desired method (see, e.g., Osman et al., Proc. Nat. Acad. Sci. (USA), 94, 14677-82 (1997)).

The inventive transgenic animals are useful for any use to which animals can be put, and they can be any desired species (e.g., pigs, cows, mice, cats, dogs, etc.). Transgenic mice in which a reporter gene is operably linked to the α1-3 galactosyltransferase promoter are valuable reagents for assessing the activity and specificity of the promoter. Transgenic livestock (e.g., pigs, cows, goats, and the like) having an inventive expression cassette in which a growth hormone is expressed under the control of the α1-3 galactosyltransferase promoter can be matured or bulked better than commonly employed strains. Tissue obtained from a transgenic animal according to the present invention can be implanted into a host according to standard surgical methods, and the invention concerns a method of xenotransplantation from a transgenic animal as described herein. The invention also provides a transgenic organ consisting essentially of transgenic cells engineered as described above (e.g., a lung, a heart, a liver, a pancreas, a stomach, an intestine, a kidney, a cornea, skin, etc.), particularly for use in the method of transplantation. The host can be any animal host, such as a pig, a dog, a cat, a cow, a goat, etc. Of course, the recipient can be a human as well, in which case the source animal preferably is a pig.

Transgenic animals lacking a functional α1-3 galactosyltransferase gene are attractive sources of organs and tissues for xenotransplantation into primates, especially humans, because the tissues of such animals lack the highly antigenic gal-□α-gal epitope. Similarly, transgenic pigs having a recombinant expression cassette in which a coding sequence for Type I fucosyltransferase, a Type II fucosyltransferase (especially α(1,2) fucosyltransferase), an α 2-3 sialyltransferase, or an α 2-6 sialyltransferase is operably linked to the α1-3 galactosyltransferase promoter also are suitable sources of xenotransplantation tissues, as the these encoded enzymes compete for the same substrate as α1-3 galactosyltransferase, and their presence can reduce (preferably below an antigenic threshold) the gal-α-gal antigens in tissues derived from such animals. Indeed, α(1,2) fucosyltransferase converts this substrate into the universally-tolerated H antigen (i.e., the “O” blood-type antigen) and also blocks the addition of the α1,3 gal moiety. As such, a gene encoding α(1,2) fucosyltransferase is an especially preferred polynucleotide for expression to be included within the inventive recombinant expression cassette. A preferred source animal for xenotransplantation tissues (and by extension the tissues themselves) preferably contains a disruption in the α1-3 galactosyltransferase gene as well as having a recombinant expression cassette in which a coding sequence for Type I fucosyltransferase, a Type II fucosyltransferase (especially α(1,2) fucosyltransferase), an α 2-3 sialyltransferase, or an α 2-6 sialyltransferase is operably linked to the α1-3 galactosyltransferase promoter. More preferably, the animal contains a disruption in the native promoter of α1-3 galactosyltransferase and an α(1,2) fucosyltransferase coding sequence under the control of its own promoter. Most preferably, the source animal also expresses exogenous human complement regulatory proteins, as discussed above, to further minimize host resistance of the xenograft tissue.

It will be apparent that a transgenic animal created in accordance with the invention can have the exogenous gene cloned in place of the native α1,3 galactosyltransferase gene (i.e., a “knock-in” approach). Indeed, in many embedment such a “knock-in’ approach is preferable, for example to avoid the potential of the development of congenital cataracts in purely “knock-out” animals (e.g., as a result of opportunistic infections of microbes bearing the gal-α-gal motif). Indeed, such an approach can afford a safe alternative to broadband antibiotics in livestock and pets, a current public health concern. In this respect, the invention can be employed to create heartier and healthier livestock and pets.

While one of skill in the art is fully able to practice the instant invention upon reading the foregoing detailed descriptions, in conjunction with the drawing and the sequence listing, the following examples will help elucidate some of its features. In particular, these examples indicate how the genomic structure of the porcine α1-3 galactosyltransferase gene is elucidated, and how the identity and activity of the α1-3 Galactosyltransferase promoter is assessed. As these examples are presented for purely illustrative purposes, they should not be used to construe the scope of the invention in a limited manner, but rather should be seen as expanding upon the foregoing description of the invention as a whole.

Many experiments described in these examples employed well known techniques and reagents (see, e.g., Sambrook et al., Molecular Cloning: A Laboratory Manual, 2d edition, Cold Spring Harbor Press (1989)). Accordingly, in the interest of brevity, the examples to not present the experimental protocols in detail. In the experiments, enzymatic isolation and culture of porcine aortic endothelial cells (PAEC) was performed. PAEC were maintained in Dulbecco's modified essential medium (DMEM) supplemented with 10% fetal bovine serum (FBS), 10,000 units of Heparin (ELKINS-SINN, Inc., Cherry Hill, N.J.), 15 mg of endothelium growth supplement (Collaborative Biomedical Product Inc., Bedford, Mass.), L-glutamine, and penicilin-streptmycin. RNA was obtained from the organs of pigs (Brain, Heart, Spleen, Gut, and Thymus) and PAEC using Trizol reagent (Gibco Ltd.,). Primers used to clone and identify regions of the porcine, murine, human, and Rhesus monkey genes are set forth at SEQ ID NOs: 45-96.

### EXAMPLE 1

This example describes the identification of the 5′ untranslated region and genomic structure of the porcine α1-3 galactosyltransferase gene.

A comparison of published sequences for the α1-3 galactosyltransferase cDNA (Hoopes et al., supra, Katayama et al., supra; Sandrin et al., supra; and Strahan et al., supra) revealed a divergence in the 5′ boundary. Some of these cDNA contain putative 5′ untranslated sequences that bear a high (>70%) homology to murine sequences identified as the second exon, and it was hypothesized that this region is conserved as an exon in the porcine genome as well.

Further 5′ sequence was cloned using 5′ RACE, and the putative transcription initiation site was probed by S1 protection assay, using standard protocols. Briefly, a plasmid containing the upstream genomic sequence was digested with restriction enzyme, Pml I, and linearized. The DNA was phosphorylated with shrimp alkaline phosphotase, heated to inactivate the enzyme, and then precipitated with ethanol. The linearized plasmid was digested again with Bgl II to yield a probe fragment, which was then end-labeled with α-³²P-ATP.

The probe was purified using G-25 sephadex, and about 16 μl was mixed with 20 μg of total RNA from pig aortic endothelial cells (PAEC), pig brain, and yeast (control), and the aliquots were coprecipitated using NH₄OAc and ethanol. Pellets were resuspended in a standard hybridyzation buffer, heated to 95° C. for 3-4 minutes, and then incubated at 42° C. overnight.

After incubation, the yeast sample was split into two aliquots, and to each was added a standard S1 nuclease buffer. S1 nuclease was added to one aliquot, while the other did not receive the enzyme. The PAEC and brain samples each received the enzyme and the buffer. All samples were incubated for 30 minutes at 37° C., after which the reactions were stopped by the addition of a standard S1 inactivation buffer. Following the reaction, the samples were then precipitated, resuspended in 5 μl of a standard gel loading buffer, and resolved using a 6% denaturing polyacrylamide gel.

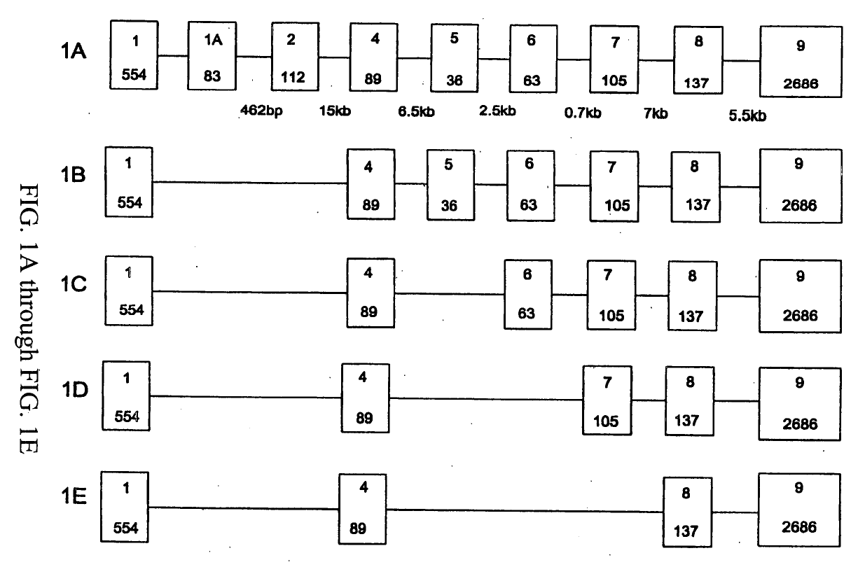

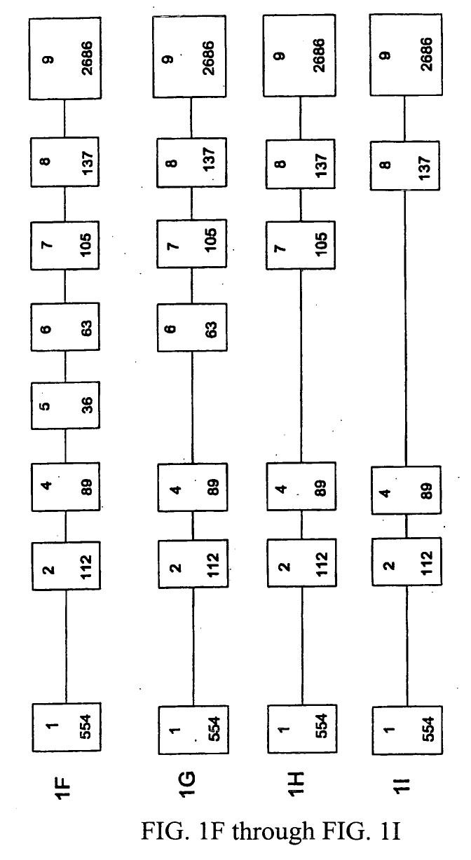

The data revealed at least 8 separate alternatively spliced transcripts from PAEC, and additional splicing patterns from brain transcripts. Analysis of these sequences revealed three potential upstream exons (1, 1A, and 2), the boundaries of which comply with the AG-GT consensus, and six coding exons (4-9) also were identified, which agreed with published results. Interestingly, the pig sequence seemingly lacks upstream exon 3 of the mouse 5′ untranslated region. The overall organization of the pig genome is depicted in FIG. 1. Alternatively spliced forms isolated from PAED are indicated in FIGS. 1B though 1I. Exon 1A is observed in transcripts isolated from brain tissue.

As mentioned, the transcripts obtained from PAEC and brain revealed several alternative splicing patterns. Using the genomic clone, intronic sequences were identified by “gene walking” using the method and reagents supplied with the Universal Geneomewalker™ Kit (Clontech Labs., Inc.). Primers (Seq ID NOs:41-56) were designed to hybridize with the cDNA, and also to the adapter sequence supplied with the Clonetech kit. A series of nested PCR reactions was then performed to clone SEQ ID NOs:7-16, which were sequenced. From these results, the intron/exon boundaries were elucidated.

Summing the nucleotides of all identified exons predicts a transcript of about 3.8 kb. This prediction was assessed by Northern analysis. 20 μg of total RNA from PAEC, and pig brain, heart, spleen, gut, and thymus, were respectively separated on formamide agarose gels, and electrotransferred onto nylon membrane. The blots were hybridized with radiolabeled probes (2.5-4.0×10⁴ cpm/ml) specific for pig GT exon 1 and exon 9 identified. The blots were exposed to Bio-MAX films (Eastman Kodak Co., Rochester, N.Y.) for 6 days with intensifying screen. The results revealed primary transcripts of between 3.5-3.8 kb, in accordance with the predicted size and the published size for the bovine transcript.

### EXAMPLE 2

This example describes the identification of the 5′ untranslated region and organization of the murine α1-3 galactosyltransferase gene.

To identify the 5′ and 3′ ends of α1,3GT gene transcripts, 5′- and 3′-RACE procedures were performed using the Marathon cDNA Amplification Kit (Clontech) with the spleen poly A⁺ RNA of Balb/C adult male as template. To identify exon-intron boundaries or 5′- and 3′-flanking region of the transcripts, Murine GenomeWalker libraries were constructed using the Universal GenomeWalker Library Kit (Clontech) with Balb/C genomic DNA.

The results of these experiments revealed several genomic sequences, which are set forth at SEQ ID NOs: 17-25. The deduced 5′ untranslated nucleotide sequences are longer by 56 bp than previously reported (Joziasse et al., J. Biol. Chem., 267,5534-41 (1992). The relative intensity of Luciferase activity by the pGL3/1280 construct was 15-fold higher than that of pGL3-Basic. The 3′-RACE revealed an extended 3′-UTR sequence 30 bp more than previously reported (Id.), but no other 3′ UTR exon usage. The overall length of the transcript was 2586 bp, 89 bp longer than previously reported (Id.).

An overall comparison of 5′-UTR of cDNA sequences of the porcine (747 bp) and murine (492 bp) α1,3GT gene indicates that the homology is observed only in the region of exon 2 (71.7%). Exon 3 observed in mice is not observed in the pig. Murine exon 1 shows no homology with porcine exon 1.

### EXAMPLE 3

This example describes the identification of the organization of the human and Rhesus monkey α1-3 galactosyltransferase untranslated pseudogene.

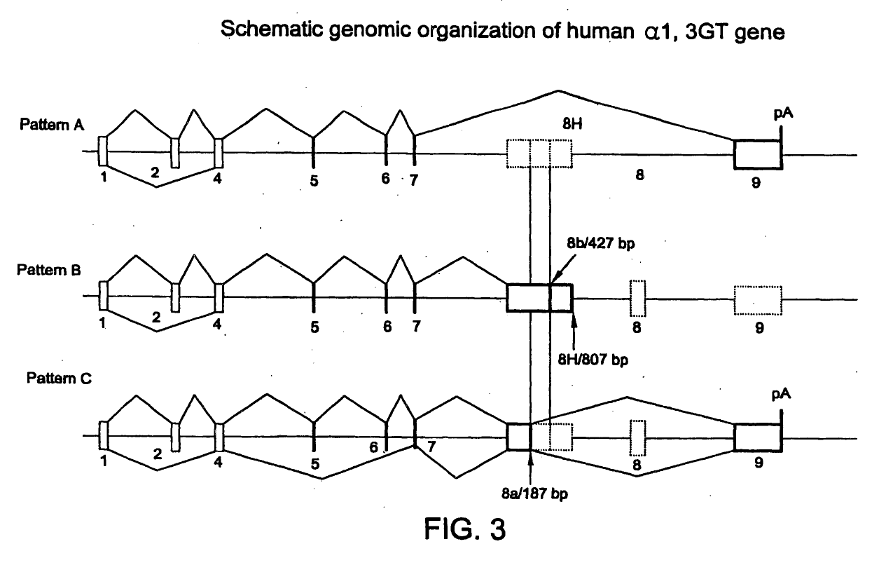

Working from published partial sequence of the human α1,3 GT ninth exon, primers were designed to identify the start and end of the gene by 5′-RACE, 3′RACE and rtPCR, as described above. Several alternate transcripts were identified, and these are set forth as SEQ ID NOs:27-34. The sequences were compared to those of other species employing a formula based on the consensus motif of the splicing acceptor junction: total number of pyramidines plus 1 (for a branched A) among forty nucleotides per junction. Intron exon boundaries were confirmed as discussed above (see SEQ ID NOs: 35-42). The organization of the alternative splicing patterns observed is indicated in FIG. 3.

Using similar techniques, primers were designed based on a partial published sequence (Genbank Accession No. M73306) having homology to exon 9. Initially, 3′RACE showed only poly-A tails, evidence that transcripts exist. 5′-RACE results revealed sequences of high homology to those α1,3 sequences previously identified (e.g., porcine, bovine and murine), consistent with the identity of the sequence as the Rhesus pseudogene. The sequence of the Rhesus monkey transcripts are ser forth at SEQ ID NOs: 43 and 44.

### EXAMPLE 4

This example describes the identification of the porcine, murine, and bovine α1-3 galactosyltransferase promoters.

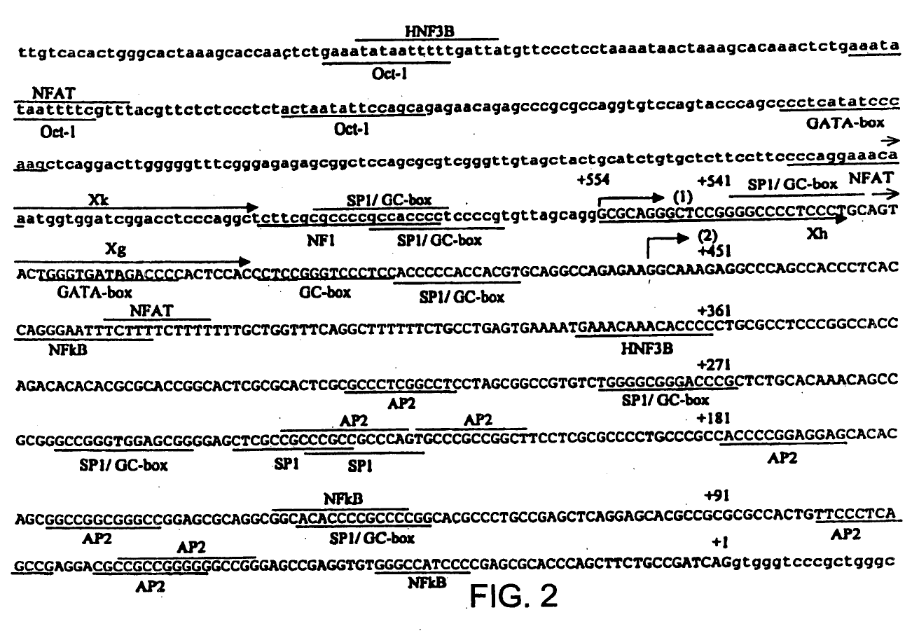

Using PCR and restriction digestions, various sized fragments between nucleotides 1981 and 2992 of SEQ ID NO:7 (porcine) and between nucleotides 375 and 1325 (murine) were generated. The fragments were cloned into a plasmid such that they were operably linked to a luciferase coding sequence. PAEC were then transfected with these constructs and probed for luciferase activity, along with a positive and a negative (no promoter) control. All fragments exhibited significantly greater promoter activity over the negative control (between about 15% and 90% relative light units, as compared to the positive control, the negative control exhibiting no luciferase activity). These results indicate that the regions are promoters and that the 5′-RACE results discussed in Examples 1 and 2 most likely represent the potential transcription initiation site (TIS). Moreover, sequence analysis of these regions reveals the presence of at least 8 SP1 or GC boxes within it and potentially seven AP-2 consensus binding motifs see also FIG. 2). This suggests that the gene may contain alternative start sites, and that sequences within exon 1 may also contain promoter activity. Other sequences from which α1,3 GT promoters can be derived are set forth as SEQ ID NOs: 1-6.

All of the references cited herein, including patents, patent applications, and publications, are hereby incorporated in their entireties by reference.

While this invention has been described with an emphasis upon preferred embodiments and illustrative examples, it will be obvious to those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

What is claimed is:

1. A recombinant expression cassette comprising an α1-3 galactosyltransferase promoter operably linked to a polynucleotide for expression, other than a polynucleotide encoding α1-3 galactosyltransferase.

2. The recombinant expression cassette of claim 1, wherein said polynucleotide for expression encodes a protein.

3. The recombinant expression cassette of claim 2, wherein said protein is a fucosyltransferase, a galactosyltransferase, a β-acetylgalactosaminyltransferase, an N-acetylglycosaminyltransferase, an N-acetylglucosaminyltransferase, a sialyltransferase, or a sulfotransferase.

4. The recombinant expression cassette of claim 2, wherein said protein is a Type I fucosyltransferase, a Type II fucosyltransferase, an α 2-3 sialyltransferase, or an α 2-6 sialyltransferase.

5. A recombinant mutating cassette comprising a first region of homology to an α1-3 galactosyltransferase genomic sequence adjacent to either a second region of homology to said α1-3 galactosyltransferase genomic sequence or a polynucleotide for insertion.

6. The recombinant mutating cassette of claim 5, comprising first and second regions of homology to an α1-3 galactosyltransferase genomic sequence flanking a polynucleotide for insertion.

7. The recombinant mutating cassette of claim 5, wherein a region of homology is homologous to an exon, an intron, or a promoter of said α1-3 galactosyltransferase genomic sequence.

8. The recombinant mutating cassette of claim 5, wherein said polynucleotide for insertion comprises an expression cassette.

9. A vector comprising the recombinant cassette of claim 1.

10. A transgenic cell harboring the vector of claim 9.

11. A vector comprising the recombinant cassette of claim 5

12. A transgenic cell harboring the vector of claim 11.

13. A chromosome comprising the recombinant cassette of claim 1.

14. A transgenic cell harboring the chromosome of claim 13.

15. The transgenic cell of claim 14, wherein said α1-3 galactosyltransferase promoter is native to said cell.

16. The transgenic cell of claim 14, wherein said polynucleotide for expression displaces a native polynucleotide encoding α1-3 galactosyltransferase.

17. The transgenic cell of claim 14, which is an embryonic stem cell, an ovum, a primordial germ cell, a spermatozoon, or a zygote.

18. The transgenic cell of claim 14, which expresses said polynucleotide for expression.

19. The cell of claim 18, wherein said polynucleotide for expression encodes a Type I fucosyltransferase, a Type II fucosyltransferase, an α 2-3 sialyltransferase, or an α 2-6 sialyltransferase, and wherein said cell produces said protein.

20. The transgenic cell of claim 14, wherein said cell produces a heterogenic complement regulatory protein (CRP).

21. An embryo consisting essentially of transgenic cells according to claim 14

22. An organ consisting essentially of transgenic cells according to claim 14.

23. A transgenic animal consisting essentially of transgenic cells according to claim 14.

24. The transgenic animal of claim 23, which is a cattle, a mouse, a pig, a cat or a dog.

25. A chromosome comprising the recombinant cassette of claim 5.

26. A transgenic cell harboring the chromosome of claim 25.

27. An embryo consisting essentially of transgenic cells according to claim 26

28. An organ consisting essentially of transgenic cells according to claim 26.

29. A transgenic animal consisting essentially of transgenic cells according to claim 26.

30. The transgenic animal of claim 29, which is a cattle, a mouse, a pig, a cat or a dog.

31. A transgenic knockout animal comprising a homozygous disruption in an endogenous α1-3 galactosyltransferase gene, wherein said disruption prevents the expression of a functional α1-3 galactosyltransferase protein.

32. The transgenic knockout animal of claim 31, wherein cells isolated from said knockout animal exhibit an increased time of survival in the presence of human serum relative to comparable cells isolated from an animal having a wild type α1-3 galactosyltransferase gene.

33. The transgenic knockout animal of claim 31, wherein the insertion replaces DNA at the start of the coding region of said α1-3 galactosyltransferase protein.

34. The transgenic knockout animal of claim 31, wherein the insertion replaces the promoter of said wild type α1-3 galactosyltransferase gene.

35. The transgenic knockout animal of claim 31, which produces at least one human protein selected from the group of proteins consisting of α1-3 galactosyltransferase, α(1,2) fucosyltransferase, and complement regulatory proteins.

36. The transgenic knockout animal of claim 31, which is a pig.
